(19) United States
(12) Patent Application Publication
(10) Pub. No.: US 2009/0198081 A1
(43) Pub. Date: Aug. 6, 2009

(54) PROCESS FOR THE AMMOXIDATION OF PROPANE AND ISOBUTANE

(76) Inventors: CHRISTOS PAPARIZOS, Willoughby, OH (US); Bhagya Chandra Sutradhar, Aurora, IL (US); Michael J. Seely, Naperville, IL (US); James F. Brazdil, JR., Glen Ellyn, IL (US); Alakananda Bhattacharyya, Glen Ellyn, IL (US)

Correspondence Address: RENNER KENNER GREIVE BOBAK TAYLOR & WEBER, FIRST NATIONAL TOWER FOURTH FLOOR, 106 S. MAIN STREET, AKRON, OH, 44308, US

(21) Appl. No.: 12/025,794

(22) Filed: Feb. 5, 2008

(51) Int. Cl. C07C 253/24

(52) U.S. Cl. 558/319

(57) ABSTRACT

A process for the ammoxidation of a saturated or unsaturated hydrocarbon to form an unsaturated nitrile, the process including the steps of contacting the hydrocarbon with ammonia, an oxygen-containing gas, and steam, in the presence of a mixed oxide catalyst.

### BACKGROUND OF THE INVENTION

1. Field of the Invention

The present invention generally relates to a process for the ammoxidation or oxidation of a saturated or unsaturated hydrocarbon to produce an unsaturated nitrile. The invention particularly relates to a process for the gas-phase conversion of propane to acrylonitrile and isobutane to methacrylonitrile (via ammoxidation).

2. Description of the Prior Art

Processes have been described for the conversion of propane to acrylonitrile and isobutane to methacrylonitrile (via an ammoxidation reaction). The art known in this field includes numerous patents and patent applications, including for example, U.S. Pat. Nos. 5,750,760, 6,036,880, 6,043,186, 6,143,916, 6,514,902, U.S. Patent Application Nos. US 2003/0088118 A1, 2004/0063990 A1, and PCT Patent Application No. WO 2004/108278 A1.

U.S. Pat. No. 3,993,680 describes a process for the ammoxidation of olefins wherein it is suggested that the feed stream may contain steam. The use of steam in oxidative processes is well known, as described in U.S. Pat. Nos. 6,982,343, 6,989,460, 7,009,075, 7,018,951, 7,026,506 and others. However, previous attempts to add steam to an alkane ammoxidation process employing a VSbSn type catalyst have shown a decrease in alkane conversion and selectivity to ammoxidation products (U.S. Pat. No. 5,686,381).

Processes that produce higher yield of desired product would be desirable. Also desirable would be processes that can operate at more efficient feed ratios (i.e. less waste of reactants and reduced production costs in terms of material and reactor capacity).

### SUMMARY OF THE INVENTION

In one embodiment, the invention includes a process for the ammoxidation of a saturated hydrocarbon or mixture of saturated and unsaturated hydrocarbon to produce an unsaturated nitrile, said process comprising contacting the saturated hydrocarbon or mixture of saturated and unsaturated hydrocarbon with a feed stream that comprises ammonia, steam, and an oxygen-containing gas, in the presence of a catalyst that includes a composition defined by the empirical formula:

Mo₁VₐSb_{b}Nb_{c}X_{d}LₑOₙ

wherein X is selected from the group consisting of W, Te, Ti, Sn, Ge, Zr, Hf, and mixtures thereof; L is selected from the group consisting of Ce, La, Pr, Nd, Sm, Eu, Gd, Tb, Dy, Ho, Er, Tm, Yb, Lu and mixtures thereof; 0.1≦a≦1.0, 0.01≦b≦1.0, 0.001≦c≦0.25, 0≦d≦0.6, 0≦e<0.04; n is the number of oxygen atoms required to satisfy valance requirements of all other elements present in the mixed oxide, with the proviso that one or more of the other elements in the mixed oxide can be present in an oxidation state lower than its highest oxidation state, a, b, c, d and e represent the molar ratio of the corresponding element to one mole of Mo.

The present invention also relates to a process for preparing an unsaturated nitrile, the process comprising providing a reactor that includes a reaction zone, one or more feed inlets for feeding a reactor feed stream into the reaction zone, a first entrance zone of the reaction zone, and an outlet for discharging reaction products and unreacted reactants, introducing a reactor feed stream into the reaction zone, said reactor feed stream comprising a saturated hydrocarbon or mixture of saturated and unsaturated hydrocarbon, ammonia, an oxygen-containing gas, and steam, and wherein the reaction zone contains a catalyst that includes a composition defined by the empirical formula:

Mo₁VₐSb_{b}Nb_{c}X_{d}LₑOₙ

wherein X is selected from the group consisting of W, Te, Ti, Sn, Ge, Zr, Hf, and mixtures thereof; L is selected from the group consisting of Ce, La, Pr, Nd, Sm, Eu, Gd, Tb, Dy, Ho, Er, Tm, Yb, Lu and mixtures thereof; 0.1≦a≦1.0, 0.01≦b≦1.0, 0.001≦c≦0.25, 0≦d≦0.6, 0≦e<0.04; n is the number of oxygen atoms required to satisfy valance requirements of all other elements present in the mixed oxide, with the proviso that one or more of the other elements in the mixed oxide can be present in an oxidation state lower than its highest oxidation state, a, b, c, d and e represent the molar ratio of the corresponding element to one mole of Mo.

### DETAILED DESCRIPTION OF THE INVENTION

The present invention generally relates to a process for the ammoxidation of a saturated or unsaturated hydrocarbon, and catalyst compositions that may be used in the process. Such processes are effective for the ammoxidation of propane to acrylonitrile and isobutane to methacrylonitrile.

In one or more embodiments, unsaturated nitrile is prepared by a process including the ammoxidation of a saturated or unsaturated or mixture of saturated and unsaturated hydrocarbon, and includes the step of contacting the hydrocarbon with ammonia, an oxygen-containing gas, and steam, in the presence of a mixed metal oxide catalyst or catalyst mixture.

### Conversion of Propane and Isobutane Via Ammoxidation Reaction

In one or more embodiments, propane is converted to acrylonitrile or isobutane to methacrylonitrile, by providing a suitable catalyst in a gas-phase flow reactor, and contacting the catalyst with propane or isobutane in the presence of oxygen, ammonia, and steam under reaction conditions effective to form acrylonitrile or methacrylonitrile. Thus, in one or more embodiments, the feed stream comprises propane, isobutane, or mixtures thereof, an oxygen-containing gas, ammonia, and steam.

The specific design of the gas-phase flow reactor is not narrowly critical. Hence, the gas-phase flow reactor can be a fixed-bed reactor, a fluidized-bed reactor, or another type of reactor. The reactor can be a single reactor, or can be one reactor in a multi-stage reactor system. In one or more embodiments, the reactor comprises one or more feed inlets for feeding a reactor feed stream to a reaction zone of the reactor, a reaction zone comprising the mixed metal oxide catalyst, and an outlet for discharging reaction products and unreacted reactants. In one or more embodiments, the portion of the reactor where one or more feed stream components first enter the reaction zone may be referred to as a first entrance zone. The reactor may further comprise one or more subsequent entrance zones where additional feed stream components enter the reaction zone. In one or more embodiments, steam is introduced in the first entrance zone of the reactor. In one or more embodiments, at least a portion of the steam fed into the reactor is introduced into the first entrance zone of the reactor.

It will be understood that one or more of the components of the feed stream may be pre-mixed, or may be added through the same feed inlet. In one embodiment, the addition of ammonia to the reaction zone may be staged, i.e. the ammonia may be added to the reactor via two or more feed inlets in different positions in the reaction zone. An example of a method for producing a nitrile wherein at least a part of the total amount of ammonia is supplied separately to a downstream position of the catalyst layer in the reactor is described in U.S. Pat. No. 5,534,650, which is incorporated herein by reference.

The reaction conditions are controlled to be effective for converting the propane to acrylonitrile or for converting the isobutane to methacrylonitrile. Generally, reaction conditions include a temperature ranging from about 300° C. to about 550° C., in one embodiment from about 325° C. to about 520° C., in some embodiments from about 350° C. to about 500° C., and in certain embodiments from about 400° C. to about 480° C. Advantageously, in one or more embodiments the process of the present invention operates effectively at higher temperatures than those of similar processes where steam is not added to the reactor. That is, in certain embodiments, the temperature of the reaction may be increased, with a corresponding increase in alkane conversion and no loss of selectivity to nitrile products. In one or more embodiments, the temperature of the reaction is at least about 400° C., in other embodiments, at least about 420° C., in another embodiment, at least about 430° C., in still another embodiment, at least about 440° C., and in yet another embodiment, at least about 450° C.

The pressure of the reaction zone can be controlled to range from about 0 psig to about 200 psig, preferably from about 0 psig to about 100 psig, and in some embodiments from about 0 psig to about 50 psig.

Generally, the flow rate of the alkane-containing feed stream through the reaction zone of the gas-phase flow reactor can be controlled to provide a weight hourly space velocity (WHSV) ranging from about 0.02 to about 5, in some embodiments from about 0.05 to about 1, and in other embodiments from about 0.1 to about 0.5, in each case, for example, in grams propane or isobutane to grams of catalyst per hour.

As stated hereinabove, the feed stream includes an oxygen-containing gas. In one or more embodiments, the molar ratio of propane or isobutane to oxygen in the feed stream is from about 0.125 to about 5, in another embodiment, from about 0.25 to about 4.5, and in another embodiment, from about 0.35 to about 4.

In one or more embodiments, the molar ratio of propane or isobutane to steam in the feed stream is from about 0.3 to about 4, and in another embodiment, from about 0.5 to about 3. In one or more embodiments, where water or water vapor is present in the alkane, ammonia, oxygen-containing gas, or other component of the feed stream, this may be taken into account when calculating the desired molar ratio of propane or isobutane to steam.

In one or more embodiments, the feed stream further includes as a diluent nitrogen or other gas that is substantially inert to the ammoxidation reaction. It will be understood that the diluent gas may be useful, i.e. in adjusting the space velocity through the reaction zone. In one or more embodiments, air is employed in the feed stream, and thus includes an oxygen-containing gas and a diluent gas. In certain embodiments, the amount of nitrogen or inert gas may be reduced based upon the amount of steam in the feed stream, so that the maximum volumetric flow capacity of the reaction system will not be exceeded. In one or more embodiments, an amount of the diluent gas may be substituted by steam.

In one or more embodiments, the molar ratio of propane or isobutane to ammonia is from about 0.3 to about 4, and in another embodiment, from about 0.5 to about 3. In one embodiment, the molar ratio of propane or isobutane to ammonia is from about 0.3 to about 2, in another embodiment, from about 0.4 to about 1.8, and in another embodiment, from about 0.5 to about 1.5.

It will be understood that the amount of ammonia in the feed stream can affect the amount of nitrile products that are formed in the reactor. Without ammonia in the feed stream, oxygen-containing products such as acids would predominate. However, too much ammonia in the feed stream may also be undesirable.

As is known in the art, commonly only a portion of the ammonia in the feed stream reacts with the alkane to produce a nitrile. In one or more embodiments, a portion of the ammonia reacts with oxygen (i.e. burns) to form oxides or other products. The amount of ammonia that reacts with oxygen to form products other than nitrites may be referred to as ammonia burn.

In these or other embodiments, a portion of the ammonia fed into the reactor remains unreacted, and exits the reactor as part of the effluent. The amount of ammonia in the effluent may be referred to as ammonia breakthrough. Typically, this unreacted ammonia is neutralized to prevent unwanted reactions downstream of the reactor, or corrosion of the reactor or recovery system. An excessive amount of ammonia breakthrough can add to production costs by requiring additional equipment for treatment. Often, however, reducing the amount of ammonia in the feed stream reduces the yield of nitrile products. The relative amount of ammonia in the feed stream that is used to produce nitrile products may be referred to as ammonia efficiency, or ammonia utilization.

Ammonia utilization may be expressed in various terms. One way of expressing ammonia utilization is to calculate a yield of nitrile products based upon the amount of ammonia fed into the reactor.

Ammonia utilization may be affected by a number of factors in addition to the ratio of ammonia in the feed stream relative to other reactants, including reaction temperature and pressure, and catalyst composition. Advantageously, it has been discovered that the addition of steam to the feed stream improves the ammonia utilization, with a corresponding decrease in ammonia breakthrough, as further described and shown hereinbelow.

The feed stream can also comprise one or more additional feed components, including acrylonitrile or methacrylonitrile product (e.g., from a recycle stream or from an earlier-stage of a multi-stage reactor). For example, the feed stream can comprise about 5% to about 30% by weight additional feed components, relative to the total amount of the feed stream, or by mole relative to the amount of propane or isobutane in the feed stream. In one embodiment, the ammoxidation process described herein is a once-through process, i.e., it operates without recycle of recovered but unreacted feed materials.

The resulting acrylonitrile or methacrylonitrile product can be isolated, if desired, from other side-products and/or from unreacted reactants according to method known in the art.

### Mixed Oxide Catalyst Composition

The improved process of the present invention has application for a number of mixed oxide ammoxidation catalyst compositions. Optionally, a mixture of a mixed metal oxide catalyst and a performance modifier may be employed.

In one embodiment, the mixed oxide catalyst composition comprises molybdenum, vanadium, niobium, and one or both of antimony and tellurium. In one or more embodiments, the mixed oxide catalyst further includes at least one element selected from the group consisting of lanthanum, cerium, praseodymium, neodymium, samarium, europium, gadolinium, terbium, dysprosium, holmium, erbium, thulium, ytterbium, and lutetium. In certain embodiments, the catalyst composition may include at least one element selected from the group consisting of tungsten, titanium, tin, germanium, lanthanum, and hafnium. As used herein, “at least one element selected from the group . . . ” or “at least one lanthanide selected from the group . . . ” includes within its scope mixtures of two or more of the listed elements or lanthanides, respectively.

In one embodiment, the mixed oxide catalyst comprises molybdenum, vanadium, antimony and niobium, and may be defined by the empirical formula:

Mo₁VₐSb_{b}Nb_{c}X_{d}LₑOₙ

wherein X is selected from the group consisting of W, Te, Ti, Sn, Ge, Zr, Hf, and mixtures thereof,
L is selected from the group consisting of La, Ce, Pr, Nd, Sm, Eu, Gd, Th, Dy, Ho, Er, Tm, Yb, Lu and mixtures thereof,
0.1<a<1.0,
0.01<b<1.0,
0.001<c<0.25,
0<d<0.6,
0<e<0.04; and
n is number of oxygen atoms required to satisfy valance requirements of all other elements present in the mixed oxide with the proviso that one or more of the other elements in the mixed oxide can be present in an oxidation state lower than its highest oxidation state, and a, b, c, d, and e represent the molar ratio of the corresponding element to one mole of Mo.

In one or more embodiments, X may be selected from the group consisting of W, Te, Ti, Ge, Sn, Zr, Hf, and mixtures thereof. In other embodiments, X may be selected from the group consisting of W, Te, Ti, Sn, Zr, Hf, and mixtures thereof. In other embodiments of the catalyst compositions described by the above empirical formulas X is one of W, Te, Ti, or Sn. In other embodiments of the catalyst compositions described by the above empirical formulas X is W.

In one or more embodiments, L may be selected from the group consisting of La, Ce, Pr, Nd, Sm, Eu, Gd, Tb, Dy, Ho, Er, Tm, Yb, and Lu. In other embodiments of the catalyst compositions described by the above empirical formulas, L is La, L is Pr, L is Ce, L is Nd, L is Sm, L is Eu, L is Gd, L is Tb, L is Dy, L is Ho, L is Er, L is Tm, L is Yb, and L is Lu. In other embodiments of the catalyst compositions described by the above empirical formulas, L is one of Nd, Ce or Pr.

In other embodiments of the catalyst compositions described by the above empirical formulas, a, b, c, and d are each independently within the following ranges: 0.1<a, 0.2<a, a<0.3, a<0.4, a<0.8, a<1.0, 0.01<b, 0.05<b, 0.1<b, b<0.3, b<0.6, b<1.0, 0.02≦c, 0.03<c, 0.04<c, c<0.05, c<0.1, c<0.15, c<0.2, c<0.25, 0≦d, 0.001<d, 0.002<d, 0.003<d, 0.004<d, d<0.006, d<0.01, d<0.02, d<0.05, d<0.1, d<0.2, 0≦e, 0.001<e, e<0.006, e<0.01, e<0.016, e<0.02, e<0.04.

In one embodiment of the catalyst compositions described by the above empirical formulas, the catalyst may optionally contain one or more other alkali metals. In this embodiment the catalyst composition comprises a mixed oxide of the empirical formula

Mo₁VₐSb_{b}Nb_{c}X_{d}LₑLi_{f}Oₙ

wherein X, L, a, b, c, d, e, and n are previously described herein, 0≦f≦0.1, and “f” represents the molar ratio of Li to one mole of Mo.

The catalyst of the present invention may be made either supported or unsupported (i.e. the catalyst may comprise a support or may be a bulk catalyst). Suitable supports are silica, alumina, zirconia, titania, or mixtures thereof. However, when zirconia or titania are used as support materials then the ratio of molybdenum to zirconium or titanium increases over the values shown in the above formulas, such that the Mo to Zr or Ti ratio is between about 1:1 to 1:10. A support typically serves as a binder for the catalyst resulting in a harder and more attrition resistant catalyst. However, for commercial applications, an appropriate blend of both the active phase (i.e. the complex of catalytic oxides described above) and the support is helpful to obtain an acceptable activity and hardness (attrition resistance) for the catalyst. Directionally, any increase in the amount of the active phase decreases the hardness of the catalyst. The support comprises between 10 and 90 weight percent of the supported catalyst. Typically, the support comprises between 40 and 60 weight percent of the supported catalyst. In one embodiment of this invention, the support may comprise as little as about 10 weight percent of the supported catalyst. In one embodiment of this invention, the support may comprise as little as about 30 weight percent of the supported catalyst. In another embodiment of this invention, the support may comprise as much as about 70 weight percent of the supported catalyst.

### Mixed Metal Oxide Catalyst Preparation

The method of making the catalyst to be used in this invention is not critical. Any method known in the art such as but not limited to hydrothermal synthesis methods and non-hydrothermal synthesis methods may be used.

In one or more embodiments, the mixed metal oxide catalyst may be prepared by the hydrothermal synthesis methods described herein. Hydrothermal synthesis methods are disclosed in U.S. Patent Application No. 2003/0004379 to Gaffney et al., Watanabe et al., “New Synthesis Route for Mo—V—Nb—Te mixed oxides catalyst for propane ammoxidation”, Applied Catalysis A: General, 194-195, pp. 479-485 (2000), and Ueda et al., “Selective Oxidation of Light Alkanes over hydrothermally synthesized Mo—V—M—O (M=Al, Ga, Bi, Sb and Te) oxide catalysts.”, Applied Catalysis A: General, 200, pp. 135-145, which are incorporated herein by reference.

In general, the catalyst compositions described herein can be prepared by hydrothermal synthesis where source compounds (i.e. compounds that contain and/or provide one or more of the metals for the mixed metal oxide catalyst composition) are admixed in an aqueous solution to form a reaction medium and the reaction medium is reacted at elevated pressure and elevated temperature in a sealed reaction vessel for a time sufficient to form the mixed metal oxide. In one embodiment, the hydrothermal synthesis continues for a time sufficient to fully react any organic compounds present in the reaction medium, for example, solvents used in the preparation of the catalyst or any organic compounds added with any of the source compounds supplying the mixed metal oxide components of the catalyst composition.

The source compounds are reacted in the sealed reaction vessel at a temperature greater than 100° C. and at a pressure greater than ambient pressure. In one embodiment, the source compounds are reacted in the sealed reaction vessel at a temperature of at least about 125° C., in another embodiment at a temperature of at least about 150° C., and in yet another embodiment at a temperature of at least about 175° C. In one embodiment, the source compounds are reacted in the sealed reaction vessel at a pressure of at least about 25 psig, and in another embodiment at a pressure of at least about 50 psig, and in yet another embodiment at a pressure of at least about 100 psig. In one or more embodiments, the source compounds are reacted in the sealed reaction vessel at a pressure of up to about 300 psig. Such sealed reaction vessels may be equipped with a pressure control device to avoid over pressurizing the vessel and/or to regulate the reaction pressure.

The source compounds may be reacted by a protocol that comprises mixing the source compounds during the reaction step. The particular mixing mechanism is not critical, and can include for example, mixing (e.g., stirring or agitating) the components during the reaction by any effective method. Such methods include, for example, agitating the contents of the reaction vessel, for example by shaking, tumbling or oscillating the component-containing reaction vessel. Such methods also include, for example, stirring by using a stirring member located at least partially within the reaction vessel and a driving force coupled to the stirring member or to the reaction vessel to provide relative motion between the stirring member and the reaction vessel. The stirring member can be a shaft-driven and/or shaft-supported stirring member. The driving force can be directly coupled to the stirring member or can be indirectly coupled to the stirring member (e.g., via magnetic coupling). The mixing is generally sufficient to allow for efficient reaction between components of the reaction medium, and to form a more homogeneous reaction medium (e.g., and resulting in a more homogeneous mixed metal oxide precursor) as compared to an unmixed reaction. This may result in more efficient consumption of starting materials and in a more uniform mixed metal oxide product. Mixing the reaction medium during the reaction step also causes the mixed metal oxide product to form in solution rather than on the sides of the reaction vessel. This allows more ready recovery and separation of the mixed metal oxide product by techniques such as centrifugation, decantation, or filtration and avoids the need to recover the majority of product from the sides of the reactor vessel. More advantageously, having the mixed metal oxide form in solution allows for particle growth on all faces of the particle rather than on the limited exposed faces when the growth occurs out from the reactor wall.

It is generally desirable to maintain some headspace in the reactor vessel. The amount of headspace may depend on the vessel design or the type of agitation used if the reaction mixture is stirred. Overhead stirred reaction vessels, for example, may take 50% headspace. Typically, the headspace is filled with ambient air which provides some amount of oxygen to the reaction. However, the headspace, as is known the art, may be filled with other gases to provide reactants like O₂ or even an inert atmosphere such as Ar or N₂. The amount of headspace and gas within it depends upon the desired reaction as is known in the art.

The source compounds can be reacted in the sealed reaction vessel at an initial pH of not more than about 4. Over the course of the hydrothermal synthesis, the pH of the reaction mixture may change such that the final pH of the reaction mixture may be higher or lower than the initial pH. In one or more embodiments, the source compounds are reacted in the sealed reaction vessel at a pH of not more than about 3.5. In some embodiments, the components can be reacted in the sealed reaction vessel at a pH of not more than about 3.0, of not more than about 2.5, of not more than about 2.0, of not more than about 1.5 or of not more than about 1.0, of not more than about 0.5 or of not more than about 0. In one or more embodiments, the pH may be from about 0.5 to about 4, in other embodiments, from about 0 to about 4, in yet other embodiments, from about 0.5 to about 3.5. In some embodiments, the pH is from about 0.7 to about 3.3, and in certain embodiments, from about 1 to about 3. The pH may be adjusted by adding acid or base to the reaction mixture.

The source compounds can be reacted in the sealed reaction vessels at the aforementioned reaction conditions (including for example, reaction temperatures, reaction pressures, pH, stirring, etc., as described above) for a period of time sufficient to form the mixed metal oxide. In one or more embodiments, the mixed metal oxide thus formed comprises a solid state solution comprising the required elements as discussed above, and at least a portion thereof includes the requisite crystalline structure for active and selective propane or isobutane oxidation and/or ammoxidation catalysts. The exact period of reaction time is not narrowly critical, and can include for example at least about three hours, at least about six hours, at least about twelve hours, at least about eighteen hours, at least about twenty-four hours, at least about thirty hours, at least about thirty-six hours, at least about forty-two hours, at least about forty-eight hours, at least about fifty-four hours, at least about sixty hours, at least about sixty-six hours or at least about seventy-two hours. Reaction periods of time can be even more than three days, including for example at least about four days, at least about five days, at least about six days, at least about seven days, at least about two weeks, at least about three weeks, or at least about one month.

Some source compounds containing and providing the metal components used in the synthesis of the catalyst (also referred to herein as a “source” or “sources”) may be provided to the reaction vessel as aqueous solutions of the metal salts. Some source compounds may be provided to the reaction vessels as solids or as slurries comprising solid particulates dispersed in an aqueous media. Some source compounds may be provided to the reaction vessels as solids or as slurries comprising solid particulates dispersed in non-aqueous solvents or other non-aqueous media.

Examples of source compounds for synthesis of the catalysts as described herein include the following. Examples of molybdenum compounds include molybdenum(VI)oxide (MoO₃), ammonium heptamolybdate and molybdic acid. Examples of lithium sources include lithium hydroxide, lithium oxide, lithium acetate, lithium tartrate, and lithium nitrate. Examples of vanadium sources include vanadyl sulfate, ammonium metavanadate, and vanadium(V)oxide. Examples of antimony sources include antimony(III)oxide, antimony(III)acetate, antimony(III)oxalate, antimony(V)oxide, antimony(III)sulfate, and antimony(III)tartrate. Examples of niobium sources include niobium oxalate, ammonium niobium oxalate, niobium oxide, niobium ethoxide and niobic acid.

Tungsten sources include ammonium metatungstate, tungstic acid, and tungsten trioxide. Tellurium sources include telluric acid, tellurium dioxide, tellurium trioxide, organic tellurium compounds such as methyltellurol and dimethyl tellurol.

Titanium sources include rutile and/or anatase titanium dioxide (TiO₂), titanium isopropoxide, TiO(oxalate), TiO(acetylacetonate)₂, and titanium alkoxide complexes, such as Tyzor 131. Titanium dioxide is available as Degussa P-25, Tronox A-K-1, and Tronox 8602 (formerly named A-K-350). Tin sources include tin(II)acetate. Germanium sources include germanium(IV)oxide. Zirconium sources include zirconyl nitrate and zirconium(IV)oxide. Hafnium sources may include hafnium(IV)chloride and hafnium(IV)oxide.

Lanthanum sources include lanthanum(III)chloride, lanthanum(III)oxide, and lanthanum(III)acetate hydrate. Cerium sources include cerium(III)chloride, cerium(III)oxide, cerium(III)isopropoxide, and cerium(III)acetate hydrate. Praseodymium sources include praseodymium(III)chloride, praseodymium(III, IV)oxide, praseodymium(III)isopropoxide, and praseodymium(III)acetate hydrate. Neodymium sources include neodymium(III)chloride, neodymium(III)oxide, neodymium(III)isopropoxide, and neodymium(III)acetate hydrate. Samarium sources may include samarium(III)chloride, samarium(III)oxide, samarium(III)isopropoxide, and samarium(III)acetate hydrate. Europium sources may include europium(III)chloride, europium(III)oxide, and europium(III)acetate hydrate. Gadolinium sources may include gadolinium(III)chloride, gadolinium(III)oxide, and gadolinium(III)acetate hydrate. Terbium sources include terbium(III)chloride, terbium(III)oxide, and terbium(III)acetate hydrate. Dysprosium sources may include dysprosium(III)chloride, dysprosium(III)oxide, dysprosium(III)isopropoxide, and dysprosium(III)acetate hydrate. Holmium sources may include holmium(III)chloride, holmium(III)oxide, and holmium(III)acetate hydrate. Erbium sources may include erbium(III)chloride, erbium(III)oxide, erbium(III)isopropoxide, and erbium(III)acetate hydrate. Thulium sources may include thulium(III)chloride, thulium(III)oxide, and thulium(III)acetate hydrate. Ytterbium sources may include ytterbium(III)chloride, ytterbium(III)oxide, ytterbium(III)isopropoxide, and ytterbium(III)acetate hydrate. Sources of lutetium may include lutetium(III)chloride, lutetium(III)oxide, and lutetium(III)acetate hydrate. Nitrates of the above listed metals may also be employed as source compounds.

The amount of aqueous solvent in the reaction medium may vary due to the solubilities of the source compounds combined to form the particular mixed metal oxide. The amount of aqueous solvent should at least be sufficient to yield a slurry (a mixture of solids and liquids which is able to be stirred) of the reactants. It is typical in hydrothermal synthesis of mixed metal oxides to leave an amount of headspace in the reactor vessel.

Following the reaction step, further steps of the catalyst preparation methods may include work-up steps, including for example cooling the reaction medium comprising the mixed metal oxide (e.g., to about ambient temperature), separating the solid particulates comprising the mixed metal oxide from the liquid (e.g., by centrifuging and/or decanting the supernatant, or alternatively, by filtering), washing the separated solid particulates (e.g., using distilled water or deionized water), repeating the separating step and washing steps one or more times, and effecting a final separating step. In one embodiment, the work up step comprises drying the reaction medium, such as by rotary evaporation, spray drying, freeze drying, or similar methods of removing liquid.

After the work-up steps, the washed and separated mixed metal oxide may be dried. Drying the mixed metal oxide can be effected under ambient conditions (e.g., at a temperature of about 25° C. at atmospheric pressure), and/or in an oven, for example, at a temperature ranging from about 40° C. to about 150° C., and in one or more embodiments at about 120° C. over a drying time ranging from about five to about fifteen hours, and in one or more embodiments of about twelve hours. Drying can be effected under a controlled or uncontrolled atmosphere, and the drying atmosphere may be an inert gas, an oxidative gas, a reducing gas or air.

In one or more embodiments of this invention, the mixed metal oxide catalyst may be prepared by non-hydrothermal synthesis methods described herein. Non-hydrothermal syntheses are also disclosed in US Patent Application No. 2006/0235238 to Satoru Komada and Sadao Shoji, and in WO 2006/019078 to Kato Takakai and Fukushima Satoshi, which are incorporated herein by reference.

One non-hydrothermal method may be generally described as follows. A first aqueous solution/slurry is prepared by combining, with heating and stirring, a molybdenum source compound, a vanadium source compound, an antimony source compound, optionally other source compounds, hydrogen peroxide, and a support sol, such as silica sol. A second aqueous solution/slurry is prepared by combining, with heating and stirring, a niobium source compound, optionally a dicarboxylic acid, and optionally other source compounds. The first and second aqueous solutions/slurries are combined to form a third aqueous solution/slurry. Precipitate and/or suspended solids may be removed, and the aqueous mixture is dried to form a dry mixed metal oxide catalyst. Various work-up steps and methods of drying and/or calcination may be employed.

In one embodiment, a non-hydrothermal method may be more specifically described as follows, where the first aqueous solution/slurry is denoted (A), and the second aqueous solution/slurry is denoted (B). Ammonium heptamolybdate, ammonium metavanadate and diantimony trioxide are added to water, followed by heating of the resultant mixture to temperatures of at least 50° C., thereby obtaining an aqueous mixture (A). It is preferred that the heating is performed while stirring the mixture. Advantageously the aqueous mixture is heated to temperatures in the range of from about 70° C. to the normal boiling point of the mixture. The heating may be performed under reflux by using equipment having a reflux condenser. In the case of heating under reflux, the boiling point generally is in the range of from about 101° C. to 102° C. Elevated temperatures may be maintained for about 0.5 hours or more. When the heating temperature is from about 80° C. to about 100° C., the heating time is typically from about 1 to about 5 hours. When the heating temperature is relatively low (e.g., lower than about 50° C.), the heating time needs to be longer.

Optionally, hydrogen peroxide and/or a sol of support material, such as silica sol, may be added to the aqueous mixture (A) after heating as described above. When hydrogen peroxide is added to the aqueous mixture (A), the amount of the hydrogen peroxide may be such that the molar ratio of hydrogen peroxide to antimony (H₂O₂/Sb molar ratio) compound in terms of antimony is in the range of from about 0.01 to about 20, in one embodiment, in the range of from about 0.5 to about 3, in another embodiment, in the range of from about 1 to about 2.5. After addition of hydrogen peroxide, aqueous mixture (A) may be stirred at temperatures in the range of from about 30° C. to about 70° C. for from about 30 minutes to about 2 hours.

In one or more embodiments, aqueous solution/slurry (B) may be formed by combining water, a niobium source compound, optionally dicarboxylic acid and/or other source compounds, with heating and stirring, thereby obtaining a preliminary niobium-containing aqueous solution or niobium-containing aqueous mixture having suspended therein a part of the niobium compound. The preliminary niobium-containing aqueous solution or niobium-containing aqueous mixture may then be cooled, whereby if a dicarboxylic acid was added, a portion of it may precipitate. The step of cooling may be followed by removing the precipitated dicarboxylic acid from the preliminary niobium-containing aqueous solution, or removing the precipitated dicarboxylic acid and the suspended niobium compound from the niobium-containing aqueous mixture, thereby obtaining a niobium-containing aqueous liquid (B).

In one embodiment, an aqueous liquid (B) may be obtained by adding a niobium compound (e.g., niobic acid) to water, followed by heating of the resultant mixture to temperatures in a range of from about 50° C. to about 100° C. Where niobic acid is the niobium source compound, a dicarboxylic acid may also be added. Dissolution of the niobium compound may be promoted by the addition of a small amount of aqueous ammonia.

Examples of suitable dicarboxylic acids include oxalic acid. In one embodiment, niobic acid and oxalic acid are added to water, followed by heating and stirring of the resultant mixture to thereby obtain an aqueous liquid (B). Generally, the molar ratio of the dicarboxylic acid to the niobium compound in terms of niobium is in the range of from about 1 to about 4, in one embodiment, in the range of from about 2 to about 4.

In other embodiments, the niobium source compound includes niobium hydrogenoxalate or ammonium niobium oxalate. When either niobium hydrogenoxalate or ammonium niobium oxalate is used as the niobium compound, the dicarboxylic acid is not required.

In general, the niobium source compound may be added in the form of a solid, a mixture, or as a dispersion in an appropriate medium. When niobic acid is used as the niobium compound, in order to remove acidic impurities with which the niobic acid may have been contaminated during the production thereof, the niobic acid may be washed with an aqueous ammonia solution and/or water prior to use. It may be advantageous to use, as the niobium compound, a freshly prepared niobium compound. However, a niobium compound that is slightly denatured (for example by dehydration) as a result of a long-term storage and the like, may be used.

The concentration of the niobium compound (in terms of niobium) in the preliminary niobium-containing aqueous solution or aqueous mixture is, in one or more embodiments, maintained within the range of from about 0.2 to about 0.8 mol/kg of the solution or mixture. The dicarboxylic acid is, in one or more embodiments, used in an amount such that the molar ratio of dicarboxylic acid to niobium compound in terms of niobium is from about 2 to about 6. When an excess amount of the dicarboxylic acid is used, a large amount of the niobium compound can be dissolved in the aqueous solution of dicarboxylic acid; however, the amount of the dicarboxylic acid that precipitates upon cooling the obtained preliminary niobium-containing aqueous solution or mixture may become too large, thus decreasing the utilization of the dicarboxylic acid. On the other hand, when an inadequate amount of the dicarboxylic acid is used, a large amount of the niobium compound may remain undissolved and suspended in the aqueous solution or mixture, and as such may be subsequently removed from the aqueous mixture, thus decreasing the degree of utilization of the niobium compound.

Any suitable method of cooling may be used. For example, the cooling can be performed simply by means of an ice bath.

The removal of the precipitated dicarboxylic acid (or precipitated dicarboxylic acid and the dispersed niobium compound) can be easily performed by conventional methods, for example, by decantation or filtration.

When the dicarboxylic acid/niobium molar ratio of the obtained niobium-containing aqueous solution is outside the range of from about 2 to about 6, either the niobium compound or dicarboxylic acid may be added to the aqueous liquid (B) so that the dicarboxylic acid/niobium molar ratio of the solution falls within the above-mentioned range. However, in general, such an operation is unnecessary since an aqueous liquid (B) having the dicarboxylic acid/niobium molar ratio within the range of from about 2 to about 4 can be prepared by appropriately controlling the concentration of the niobium compound, the ratio of the dicarboxylic acid to the niobium compound and the cooling temperature of the above-mentioned preliminary niobium-containing aqueous solution or aqueous mixture.

The aqueous liquid (B) may further comprise additional component(s). In one or more embodiments, aqueous liquid (B) may further comprise hydrogen peroxide (H₂O₂). In these or other embodiments, aqueous liquid (B) may further comprise one or more of an antimony compound (e.g. diantimony trioxide), a titanium compound (e.g. titanium dioxide, which can be a mixture of rutile and anatase forms), and a cerium compound (e.g. cerium acetate). In one embodiment, the amount of the hydrogen peroxide is such that the molar ratio of hydrogen peroxide to niobium compound (H₂O₂/Nb molar ratio) in terms of niobium is in the range of from about 0.5 to about 20, and in another embodiment, in the range of from about 1 to about 20. In certain embodiments, an antimony compound is mixed with at least a part of the aqueous liquid (B) and the hydrogen peroxide such that the molar ratio (Sb/Nb molar ratio) of the antimony compound in terms of antimony to the niobium compound in terms of niobium is not more than about 5, and in one embodiment, in the range of from about 0.01 to about 2.

Aqueous mixture (A) and aqueous liquid (B) may be mixed together in an appropriate ratio to form an aqueous solution/slurry. The ratio of (a) to (b) will be in accordance with the desired composition of the catalyst. The amount of solids in the aqueous mixture is generally in a range upward from about 10 percent by weight. In one embodiment, the amount of solids in the aqueous mixture is from about 10 to 60 percent by weight, in another embodiment, from about 15 to 55 percent by weight, and in another embodiment, the amount of solids in the mixture is from about 20 to about 50 percent by weight, based upon the total weight of the mixture.

In one or more embodiments, where a silica supported catalyst is desired, the aqueous solution/slurry is prepared so as to contain a source of silica (namely, a silica sol or fumed silica). The amount of the source of silica may be appropriately adjusted in accordance with the desired amount of the silica carrier in the catalyst to be obtained.

The aqueous solution/slurry may be dried to remove the liquid portion. Drying may be conducted by conventional methods, such as spray drying or evaporation drying. Spray drying is particularly useful, because a fine, spherical, dry solid is obtained. The spray drying can be conducted by centrifugation, by the two-phase flow nozzle method or by the high-pressure nozzle method. In one or more embodiments, heated air may be used as a heat source for drying. It may be advantageous if the temperature of the spray dryer at an entrance to the dryer section thereof is from about 150° C. to about 300° C.

At this point, the dried material, whether formed via hydrothermal or nonhydrothermal methods, may be referred to as dry mixed metal oxide catalyst. It will be understood that the terms “dry” and “dried” describe a solid from which most liquid has been removed, although some moisture may remain. Therefore, unless otherwise indicated, the terms “dry” and “dried” should be interpreted to mean substantially dry. For purposes of this specification, the term “dry mixed metal oxide catalyst” continues to refer to this substance throughout optional further treatments to which the dry mixed metal oxide catalyst may be subjected, including calcination and grinding, as described hereinbelow. Thus, a dry mixed metal oxide catalyst may be calcined or uncalcined, ground, crushed, pelleted, extruded, or otherwise formed or shaped.

As stated hereinabove, the dried mixed metal oxide catalyst may be further treated. Such treatments can include for example calcinations (e.g., including heat treatments under oxidizing or reducing conditions) effected under various treatment atmospheres. The dry mixed metal oxide can be crushed or ground prior to such treatment, and/or intermittently during such treatment. In one embodiment, for example, the dry mixed metal oxide can be optionally crushed, and then calcined.

The calcination may be effected in an inert, reducing, or oxidizing atmosphere. In one embodiment, at least a part of the calcination is conducted in an atmosphere of an inert gas (e.g., under a flow of an inert gas), such as nitrogen gas that is substantially free of oxygen. In one or more embodiments, the calcination conditions include temperatures ranging from about 200° C. to about 700° C., in other embodiments, from about 400° C. to about 650° C.

In one or more embodiments, the heating temperature of the dry mixed metal oxide catalyst is continuously or intermittently elevated from less than about 400° C. to from about 550° C. to about 700° C. In certain embodiments, multi-step calcination may be employed. In these embodiments, the dry mixed metal oxide catalyst may be partially calcined at a relatively low temperature of at least about 200° C., and then at one or more higher temperatures of at least about 400° C., within the ranges set forth hereinabove.

The treated (e.g., calcined) mixed metal oxide may be further mechanically treated, including for example by grinding, sieving and pressing the mixed metal oxide into its final form for use in the process of the present invention.

In other embodiments, the catalyst may be shaped into its final form prior to any calcinations or other heat treatment. For example, in the preparation of a fixed bed catalyst, the catalyst precursor slurry is typically dried by heating at an elevated temperature and then shaped (e.g. extruded, pelletized, etc.) to the desired fixed bed catalyst size and configuration prior to calcination. Similarly, in the preparation of fluid bed catalysts, the catalyst precursor slurry may be spray dried to yield microspheroidal catalyst particles having particle diameters in the range from about 10 to about 200 microns and then calcined. Variations on the above methods will be recognized by those skilled in the art.

Calcinations can be conducted using a rotary kiln, a fluidized-bed kiln or the like. In one or more embodiments, calcination is conducted in a non-stationary state, and problems of uneven calcination (leading to a deterioration of the properties and/or a breakage or cracking of the catalyst obtained) are avoided.

Conditions of calcination may be preselected such that the catalyst formed has a specific surface of from about 5 m²/g to about 35 m²/g. Advantageously, the conditions of calcination may be preselected such that the resulting catalyst comprises one or more crystalline phases.

In one or more embodiments, a catalyst mixture may be employed. In one embodiment, the catalyst mixture includes a physical mixture of a mixed metal oxide catalyst and a performance modifier. Generally, the performance modifier is a solid that may be physically mixed with a mixed oxide catalyst to improve catalyst performance. Advantageously, the performance modifier may be mixed with the catalyst prior to introducing the catalyst into a reactor.

Examples of performance modifiers include aluminum compounds, antimony compounds, arsenic compounds, boron compounds, cerium compounds, germanium compounds, lithium compounds, neodymium compounds, niobium compounds, phosphorus compounds, selenium compounds, tantalum compounds, tellurium compounds, titanium compounds, tungsten compounds, vanadium compounds, zirconium compounds, and mixtures thereof.

The amount of performance modifier that is added to the catalyst mixture is not particularly limited. In one embodiment, the amount of performance modifier may be expressed in terms of moles of the performance modifier per mole of molybdenum in the mixed oxide catalyst. In one or more embodiments, the catalyst mixture comprises at least about 0.01 moles performance modifier per mole of molybdenum. In these or other embodiments, the catalyst mixture comprises up to about 1.0 moles performance modifier per mole of molybdenum in the mixed oxide catalyst. In one embodiment, the catalyst mixture comprises from about 0.01 to about 1.0 moles performance modifier per mole of molybdenum. In another embodiment, the catalyst mixture comprises from about 0.011 to about 0.5, and in yet another embodiment, from about 0.012 to about 0.2 moles performance modifier per mole of molybdenum.

The dry mixed metal oxide catalyst composition may be combined with the performance modifier by physical mixing to form a catalyst mixture. Advantageously, the dry mixed metal oxide catalyst and the performance modifier may be physically mixed without the addition of liquids. In one embodiment, the performance modifier is finely ground prior to combining the modifier with the catalyst. In another embodiment, the performance modifier has a more coarse particle size, i.e. on the order of the particle size of the catalyst.

The performance modifier may be added at various stages of the preparation of the catalyst composition, to catalyst that is in its final form for use in a reactor, or to catalyst that has already seen time on-stream.

In one or more embodiments, the performance modifier is mixed with the dry mixed metal oxide catalyst prior to final calcination of the catalyst. In other embodiments, the catalyst composition is calcined as described hereinabove prior to addition of the performance modifier. In certain embodiments, the catalyst composition is mechanically treated as described hereinabove prior to addition of the performance modifier. In one embodiment, the physical mixture may be subjected to a heat treatment or calcination. Examples of catalyst mixtures are further described in U.S. Patent Application No. 60/979,276, which is incorporated herein by reference.

The catalyst mixtures described herein when employed in the single pass (i.e. no recycle) ammoxidation of propane are capable of producing a yield of about 45 percent acrylonitrile, or higher. The effluent of the reactor may also include hydrogen cyanide (HCN), acetonitrile or methyl cyanide (CH₃CN), COₓ (carbon dioxide+carbon monoxide), unreacted oxygen (O₂), ammonia (NH₃), nitrogen (N₂), helium (He), and entrained catalyst fines.

Advantageously, in one or more embodiments of the process of the present invention, the yield of acrylonitrile is improved when compared to the same process but where steam is not added to the reactor feed stream. In certain embodiments, the ammonia utilization is improved, as evidenced by an equally good yield of nitrile products at a lower ratio of ammonia in the feed stream. In one or more embodiments, the amount of ammonia breakthrough in the reactor effluent may be reduced by lowering the ratio of ammonia in the feed stream, without sacrificing the yield of nitrile products. In one or more embodiments, the alkane conversion may be increased by raising the reactor temperature, while maintaining the utilization of the ammonia and the selectivity to nitrile products.

Advantageously, in one or more embodiments of the process of the present invention, the amount of ammonia burned is decreased when compared to the same process but where steam is not added to the reactor. In one embodiment, the amount of ammonia burned is decreased by at least about 10%, in another embodiment, at least about 20%, in yet another embodiment, at least about 30%, in still another embodiment, at least about 40%, when compared to the same process but where steam is not added to the reactor, and based upon the total amount of ammonia in the reactor feed stream.

### SPECIFIC EMBODIMENTS

In order to illustrate the instant invention, samples of a mixed metal oxide catalyst composition were prepared and then evaluated under ammoxidation reaction conditions with and without steam in the feed gas mixture. The compositions listed below are nominal compositions, based on the total metals added in the preparation of the catalyst mixture. Since some metals may be lost or may not completely react during the catalyst preparation, the actual composition of the finished catalyst mixture may vary slightly from the nominal compositions shown below.

Mixed metal oxide catalyst was prepared in its final form, according to methods described herein. In Examples 3-6, a portion of the catalyst was combined with a catalyst modifier and mixed in a dry state, by using a mechanical mixer.

Catalyst was evaluated in a 40 cc fluid bed reactor having a diameter of 1-inch. The reactor was charged with about 20 to about 45 g of particulate catalyst or catalyst mixture. Propane was fed into the reactor at a rate of about 0.05 to about 0.06 WWH (i.e., weight of propane/weight of catalyst/hour). Oxygen, ammonia, steam, and nitrogen were fed into the reactor. The oxygen and nitrogen feed ratios were as follows: O₂ 3.39/C₃ 1.0/N₂ 12.61. The steam (H₂O) and ammonia (NH₃) ratios are shown in Table 1. Pressure inside the reactor was maintained at about 2 to about 15 psig. Reaction temperatures were in the range of about 430 to about 460° C.

Ammonia breakthrough was determined by titrating a sample of the reactor effluent via conventional titration techniques.

Example #1 was prepared with the nominal composition: MoV_{0.3}Sb_{0.2}Nb_{0.08}Ti_{0.1}Ce_{0.005}Oₙ and including 45% by weight silica support. Example 1-1 was run without steam in the feed gas mixture. Examples 1-2, 1-3, and 1-4 were run with steam in the feed gas mixture, at the temperatures shown in Table 1.

Example #2 was prepared with the nominal composition: MoV_{0.21}Sb_{0.24}Nb_{0.09}Oₙ and including 45% by weight silica support. Example 2-1 was run without steam in the feed gas mixture. Examples 2-2 and 2-3 were run with steam in the feed gas mixture, at the temperatures shown in Table 1.

Example #3 was prepared by physically mixing a catalyst having the nominal composition: MoV_{0.21}Sb_{0.24}Nb_{0.09}Oₙ and including 45% by weight silica support with 0.05 moles of Sb₂O₃. Example 3-1 was run without steam in the feed gas mixture. Examples 3-2 and 3-3 were run with steam in the feed gas mixture, at the temperatures shown in Table 1.

Example #4 was prepared by physically mixing a catalyst having the nominal composition: MoV_{0.21}Sb_{0.24}Nb_{0.09}Oₙ and including 45% by weight silica support with 0.1 moles of Sb₂O₃. Example 4-1 was run without steam in the feed gas mixture. Example 4-2 was run with steam in the feed gas mixture, at the temperature shown in Table 1.

Example #5 was prepared by physically mixing a catalyst having the nominal composition: MoV_{0.21}Sb_{0.24}Nb_{0.09}Oₙ and including 45% by weight silica support with 0.05 moles TiO₂. Example 5-1 was run without steam in the feed gas mixture. Example 5-2 was run with steam in the feed gas mixture, at the temperature shown in Table 1.

Example #6 was prepared by physically mixing a catalyst having the nominal composition: MoV_{0.21}Sb_{0.24}Nb_{0.09}Oₙ and including 45% by weight silica support with 0.2 moles Sb₂O₃. Example 6-1 was run without steam in the feed gas mixture. Example 6-2 was run with steam in the feed gas mixture, at the temperature shown in Table 1.

TABLE 1

| | | Molar | | | | | | % O₂ | | | | | |
|---|---|---|---|---|---|---|---|---|---|---|---|---|---|
| | | ratio to C₃H₈ | | Pressure | Temp. | % NH₃ | g/CSF NH₃ | in | C₃H₈ | Yield, % | | | AN |
| Ex | WWH | H₂O | NH₃ | psig | ° C. | Burned | Breakthrough | Effluent | Conv % | AN | HCN | AA | Selectivity % |
| 1-1 | 0.05 | 0.0 | 1.35 | 5 | 440 | 45.0 | 0.15 | 4.4 | 77.9 | 41.7 | 5.8 | 0.8 | 53.6 |
| 1-2 | 0.05 | 2.0 | 1.35 | 5 | 440 | 25.6 | 0.58 | 5.3 | 76.5 | 41.3 | 5.9 | 0.1 | 54.0 |
| 1-3 | 0.05 | 2.0 | 1.35 | 5 | 449 | 27.6 | 0.5 | 4.2 | 81.7 | 44.2 | 5.7 | 0.8 | 54.1 |
| 1-4 | 0.05 | 2.0 | 1.35 | 5 | 460 | 35.8 | 0.32 | 2.7 | 86.5 | 45.9 | 5.3 | 1.1 | 53.1 |
| 2-1 | 0.05 | 0.0 | 1.20 | 10 | 430 | 51.7 | 0.22 | 4.9 | 73.1 | 36.2 | 4.2 | 1.0 | 49.6 |
| 2-2 | 0.05 | 0.0 | 1.20 | 10 | 440 | 45.1 | 0.21 | 4.4 | 79.0 | 36.7 | 4.9 | 1.8 | 46.5 |
| 2-3 | 0.05 | 2.0 | 1.20 | 10 | 461 | 41.9 | 0.29 | 2.4 | 84.3 | 39.0 | 3.5 | 2.5 | 46.3 |
| 3-1 | 0.05 | 0.0 | 1.20 | 10 | 430 | 50.4 | 0.19 | 5.4 | 72.1 | 35.5 | 5.1 | 1.3 | 49.2 |
| 3-2 | 0.05 | 0.0 | 1.20 | 10 | 440 | 52.4 | 0.12 | 4.2 | 79.3 | 38.9 | 4.6 | 2.2 | 49.1 |
| 3-3 | 0.05 | 2.0 | 1.20 | 10 | 460 | 50.2 | 0.14 | 2.1 | 88.4 | 42.0 | 4.3 | 2.7 | 47.5 |
| 4-1 | 0.05 | 0.0 | 1.20 | 10 | 440 | 47.2 | 0.13 | 4.4 | 81.6 | 41.7 | 4.3 | 1.9 | 51.1 |
| 4-2 | 0.05 | 2.0 | 1.20 | 10 | 460 | 44.5 | 0.15 | 2.3 | 90.2 | 43.0 | 3.9 | 2.9 | 47.7 |
| 5-1 | 0.05 | 0.0 | 1.20 | 10 | 440 | 47.7 | 0.16 | 2.5 | 84.8 | 36.3 | 4.9 | 1.6 | 42.9 |
| 5-2 | 0.06 | 2.0 | 1.20 | 10 | 460 | 47.5 | 0.18 | 1.9 | 87.8 | 37.5 | 4.4 | 2.3 | 42.8 |
| 6-1 | 0.05 | 0.0 | 1.20 | 10 | 440 | 49.6 | 0.11 | 4.3 | 79.6 | 39.3 | 4.5 | 1.9 | 49.4 |
| 6-2 | 0.05 | 2.0 | 1.20 | 10 | 460 | 51.3 | 0.08 | 2.6 | 87.1 | 41.0 | 3.7 | 2.5 | 47.0 |

While the foregoing description and the above embodiments are typical for the practice of the instant invention, it is evident that many alternatives, modifications, and variations will be apparent to those skilled in the art in light of this description. Accordingly, it is intended that all such alternatives, modifications and variations are embraced by and fall within the spirit and broad scope of the appended claims.

We claim:

1. A process for the ammoxidation of a saturated hydrocarbon or mixture of saturated and unsaturated hydrocarbon to produce an unsaturated nitrile, said process comprising:
contacting the saturated hydrocarbon or mixture of saturated and unsaturated hydrocarbon with a feed stream that comprises ammonia, steam, and an oxygen-containing gas, in the presence of a catalyst that includes a composition defined by the empirical formula:
Mo₁VₐSb_{b}Nb_{c}X_{d}LₑOₙ
wherein X is selected from the group consisting of W, Te, Ti, Sn, Ge, Zr, Hf, and mixtures thereof; L is selected from the group consisting of Ce, La, Pr, Nd, Sm, Eu, Gd, Tb, Dy, Ho, Er, Tm, Yb, Lu and mixtures thereof; 0.1≦a≦1.0, 0.01≦b≦1.0, 0.001≦c≦0.25, 0≦d≦0.6, 0≦e<0.04; n is the number of oxygen atoms required to satisfy valance requirements of all other elements present in the mixed oxide, with the proviso that one or more of the other elements in the mixed oxide can be present in an oxidation state lower than its highest oxidation state, a, b, c, d and e represent the molar ratio of the corresponding element to one mole of Mo.

2. The process of claim 1, wherein the saturated hydrocarbon comprises propane, isobutane, or mixtures thereof.

3. The process of claim 1, wherein the molar ratio of saturated hydrocarbon to ammonia in the feed stream is from about 0.3 to about 4.

4. The process of claim 1, wherein the molar ratio of saturated hydrocarbon to steam in the feed stream is from about 0.3 to about 4.

5. The process of claim 1, wherein the molar ratio of saturated hydrocarbon to oxygen-containing gas in the feed stream is from about 0.125 to about 5.

6. The process of claim 1, wherein said step of contacting includes occurs at a reactor temperature of from about 300° C. to about 550° C.

7. The process of claim 1, wherein 0.1<a<0.8, 0.01<b<0.6, 0.001<c<0.3, and 0.001<d<0.1.

8. The process of claim 1, wherein said catalyst further includes a performance modifier.

9. The process of claim 8, wherein said performance modifier is selected from aluminum compounds, antimony compounds, arsenic compounds, boron compounds, cerium compounds, germanium compounds, lithium compounds, neodymium compounds, niobium compounds, phosphorus compounds, selenium compounds, tantalum compounds, tellurium compounds, titanium compounds, tungsten compounds, vanadium compounds, zirconium compounds, and mixtures thereof.

10. The process of claim 1, wherein the catalyst composition further comprises one or more alkali elements, and may be represented by empirical formula:
Mo₁VₐSb_{b}X_{c}L_{d}AₑOₙ
wherein A is Li, Na, K, Cs, Rb or a mixture thereof, 0≦e≦0.1, and e represents the molar ratio of the corresponding element to one mole of Mo.

11. The process of claim 1, wherein the catalyst composition comprises a support selected from the group consisting of silica, alumina, zirconia, titania, or mixtures thereof.

12. The process of claim 1, wherein the support comprises about 10 to about 70 weight percent of the catalyst.

13. The process of claim 1, wherein the oxygen-containing gas further comprises a diluent gas.

14. The process of claim 13, wherein an amount of the diluent gas is substituted by steam.

15. A process for preparing an unsaturated nitrile, the process comprising:
providing a reactor that includes a reaction zone, one or more feed inlets for feeding a reactor feed stream into the reaction zone, a first entrance zone of the reaction zone, and an outlet for discharging reaction products and unreacted reactants,
introducing a reactor feed stream into the reaction zone, said reactor feed stream comprising a saturated hydrocarbon or mixture of saturated and unsaturated hydrocarbon, ammonia, an oxygen-containing gas, and steam, and wherein the reaction zone contains a catalyst that includes a composition defined by the empirical formula:
Mo₁VₐSb_{b}Nb_{c}X_{d}LₑOₙ
wherein X is selected from the group consisting of W, Te, Ti, Sn, Ge, Zr, Hf, and mixtures thereof; L is selected from the group consisting of Ce, La, Pr, Nd, Sm, Eu, Gd, Tb, Dy, Ho, Er, Tm, Yb, Lu and mixtures thereof; 0.1≦a≦1.0, 0.01≦b≦1.0, 0.001≦c≦0.25, 0≦d≦0.6, 0≦e<0.04; n is the number of oxygen atoms required to satisfy valance requirements of all other elements present in the mixed oxide, with the proviso that one or more of the other elements in the mixed oxide can be present in an oxidation state lower than its highest oxidation state, a, b, c, d and e represent the molar ratio of the corresponding element to one mole of Mo.

16. The process of claim 15, wherein at least a portion of the steam is introduced into the first entrance zone.

17. The process of claim 15, wherein the saturated hydrocarbon comprises propane, isobutane, or mixtures thereof.

18. The process of claim 15, wherein the molar ratio of saturated hydrocarbon to ammonia in the feed stream is from about 0.3 to about 4.

19. The process of claim 15, wherein the molar ratio of saturated hydrocarbon to steam in the feed stream is from about 0.3 to about 4.

20. The process of claim 15, wherein the molar ratio of saturated hydrocarbon to oxygen-containing gas in the feed stream is from about 0.125 to about 5.

21. The process of claim 15, wherein said step of contacting includes occurs at a reactor temperature of from about 300° C. to about 550° C.

22. The process of claim 15, wherein a portion of the ammonia is burned during the process, and wherein the amount of ammonia that is burned is at least 10% lower than the amount of ammonia that is burned in an identical process but where no steam is added to the reactor feed stream, based upon the total amount of ammonia in the feed stream.

23. The process of claim 15, wherein the oxygen-containing gas further comprises a diluent gas.

24. The process of claim 23, wherein an amount of the diluent gas is substituted by steam.
